(19) United States
(12) Patent Application Publication
(10) Pub. No.: US 2025/0029246 A1
(43) Pub. Date: Jan. 23, 2025

(54) METHOD AND SYSTEM TO PREDICT RADIOFREQUENCY HEATING OF MEDICAL IMPLANTS

(71) Applicant: Northwestern University, Evanston, IL (US)

(72) Inventor: Laleh Golestani Rad, Wilmette, IL (US)

(21) Appl. No.: 18/779,742

(22) Filed: Jul. 22, 2024

Related U.S. Application Data
(60) Provisional application No. 63/528,202, filed on Jul. 21, 2023.

(51) Int. Cl. G06T 7/00; A61N 1/05

(52) U.S. Cl. CPC G06T 7/0012; A61N 1/0534; G06T 2207/10088; G06T 2207/20081; G06T 2207/20084; G06T 2207/30052

(57) ABSTRACT

A system to predict heating in implants includes a memory configured to store one or more phase images of a medical implant that is implanted within a patient. The one or more phase images include the medical implant and tissue surrounding the medical implant. The one or more phase images are based on an imaging pre-scan of the patient. The system also includes a processor operatively coupled to the memory and configured to analyze the one or more phase images to determine values for one or more properties of the tissue surrounding the implant. The processor also predicts, based at least in part on the analyzed one or more phase images, a temperature increase of the medical implant that will occur during a subsequent imaging scan of the patient.

### CROSS-REFERENCE TO RELATED APPLICATION

The present application claims the priority benefit of U.S. Provisional Patent App. No. 63/528,202 filed on Jul. 21, 2023, the entire disclosure of which is incorporated herein by reference.

### BACKGROUND

Magnetic resonance imaging (MRI) refers to a medical imaging technique that utilizes a magnetic field in conjunction with computer-generated radio waves to generate detailed images of the body. Magnetic resonance imaging is the preferred imaging modality for numerous neurological and cardiac disorders. Despite continuous advances in MRI, a persistent safety concern for patients with active implantable medical devices (AIMDs), such as deep brain stimulation (DBS) systems and cardiac implantable electronic devices (CIEDs), is the risk of radiofrequency (RF) heating of the tissue surrounding the conductive lead.

### SUMMARY

An illustrative system to predict heating in implants includes a memory configured to store one or more phase images of a medical implant that is implanted within a patient. The one or more phase images include the medical implant and tissue surrounding the medical implant. The one or more phase images are based on an imaging pre-scan of the patient. The system also includes a processor operatively coupled to the memory and configured to analyze the one or more phase images to determine values for one or more properties of the tissue surrounding the implant. The processor also predicts, based at least in part on the analyzed one or more phase images, a temperature increase of the medical implant that will occur during a subsequent imaging scan of the patient.

In an illustrative embodiment, the imaging pre-scan is conducted for 30 seconds or less. In another embodiment, a first amount of power used for the pre-scan is less than a second amount of power used for the subsequent imaging scan. In another illustrative embodiment, the processor predicts the temperature increase of the medical implant based at least in part on a desired power to be used during the subsequent imaging scan. The processor can also predict the temperature increase of the medical implant based at least in part on a desired duration of the subsequent imaging scan.

In one embodiment, the processor analyzes the one or more phase images to determine a specific absorption rate (SAR) of the medical implant, and the processor predicts the temperature increase based at least in part on the determined SAR. In another embodiment, the one or more properties of the tissue include a thermal conductivity of the tissue, a heat capacity of the tissue, a density of the tissue, an amount of metabolic heat generation of the tissue, and/or an amount of perfusion through the tissue. In another embodiment, the processor utilizes a neural network to analyze the one or more phase images, and the neural network includes a first hidden layer and a hidden second layer, where the first hidden layer includes 11 hidden neurons and the second hidden layer includes 9 hidden neurons.

In another embodiment, the system includes an imaging device in communication with the processor. The imaging pre-scan is performed using the imaging device, and the one or more phase images are generated by the imaging device. In another embodiment, the processor compares the predicted temperature increase of the medical implant to a temperature increase threshold, and the processor issues an alert in response to a determination that the predicted temperature increase exceeds the temperature increase threshold. In one embodiment, the processor is configured to determine, based on the analysis of the one or more phase images and a desired duration of the subsequent imaging scan, a maximum power that can be used during the subsequent imaging scan such that the temperature increase does not exceed a temperature increase threshold.

An illustrative method of predicting heating in implants includes performing, by an imaging device, an imaging pre-scan of a patient with a medical implant. The method also includes storing, in a memory of a computing device in communication with the imaging device, one or more phase images of the medical implant that is implanted within the patient. The one or more phase images include tissue surrounding the medical implant, and the one or more phase images result from the pre-scan of the patient. The method also includes analyzing, by a processor of the computing device, the one or more phase images to determine values for one or more properties of the tissue surrounding the implant. The method further includes predicting, based at least in part on the analyzed one or more phase images, a temperature increase of the medical implant that will occur during a subsequent imaging scan of the patient.

In an illustrative embodiment, the pre-scan is conducted for 30 seconds or less, and the pre-scan is conducted using a first amount of power, where the first amount of power is lower than a second amount of power used to conduct the subsequent imaging scan. In another embodiment, the method includes receiving at least one of a desired power and a desired duration for the subsequent imaging scan, where the predicted temperature increase is based on one or more of the desired power and the desired duration. In another embodiment, the one or more properties of the tissue include a thermal conductivity of the tissue. In yet another embodiment, the method further includes comparing, by the processor, the predicted temperature increase of the medical implant to a temperature increase threshold, and issuing, by the processor, an alert in response to a determination that the predicted temperature increase exceeds the temperature increase threshold.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A depicts an experimental setup using a 3 Tesla (T) Siemens Prisma scanner in accordance with an illustrative embodiment.

FIG. 1B depicts three-dimensional (3D) rendering of an anthropomorphic phantom with a DBS system in accordance with an illustrative embodiment.

FIG. 2 depicts an architecture of a proposed neural network that includes two hidden layers with 11 and 9 hidden neurons in the first and second layer, respectively, in accordance with an illustrative embodiment.

FIG. 3 depicts temporal profiles of temperature at the DBS lead-tip during RF exposure in accordance with an illustrative embodiment.

FIG. 4 depicts a distribution of ΔTₘₐₓ for 346 phantom experiments performed with a commercial DBS system in accordance with an illustrative embodiment.

FIG. 5 depicts distribution of ΔTₘₐₓ for cases in the additional test datasets including RF heating of a DBS system during MRI in an open-bore scanner and MRI at 1.5 T and of CIEDs during MRI at 1.5 T in accordance with an illustrative embodiment.

FIG. 6A depicts performance of the proposed neural network with the initial hold-out test dataset in accordance with an illustrative embodiment.

FIG. 6B depicts performance of the proposed neural network with a DBS during MRI in an open-bore scanner at 1.2 T in accordance with an illustrative embodiment.

FIG. 6C depicts performance of the proposed neural network with a DBS during MRI at 1.5 T in accordance with an illustrative embodiment.

FIG. 6D depicts performance of the proposed neural network with CIEDs during MRI at 1.5 T in accordance with an illustrative embodiment.

FIG. 7 is a block diagram of a computing system to implement a system to predict RF heating in an implant in accordance with an illustrative embodiment.

### DETAILED DESCRIPTION

A safety concern for patients with active implantable medical devices (AIMDs), such as deep brain stimulation (DBS) systems and cardiac implantable electronic devices (CIEDs), is the risk of radiofrequency (RF) heating of the tissue surrounding the conductive lead of the AIMD. To help improve the safety of these devices, there have been consistent efforts to quantify and reduce RF heating of AIMDs. The technical specification ISO/TS 10974, recognized by the United States Food and Drug Administration (FDA) as the consensus standard for magnetic resonance (MR)-Conditional devices, outlines a four-tier approach to quantify RF heating. For elongated implants, such as leads in neuromodulation or cardiac devices, recommended test procedures include application of a transfer function or full-wave electromagnetic (EM) simulations of the patient body model and the medical implant. However, there are instances when these standard methods are not feasible, such as in the presence of abandoned or broken leads, either alone or near intact leads.

Another challenge with traditional MRI techniques is that the recommended limits sent by device manufacturers to ensure the safety of the general population may be too conservative for specific cases. For example, RF heating of leads in neuromodulation and cardiac electronic implants has been shown to vary greatly between patients, with up to two orders of magnitude difference, due to the lead trajectory. Thus, a tool that can predict RF heating around the tip of an unknown lead in an individual, based on the information obtained from the first few seconds of a scan, would be highly desirable.

Recent research has employed machine learning (ML) to predict subject-specific local specific absorption rate (SAR) from B1+ maps and SAR distributions from anatomical magnetic resonance imaging (MRI) images. Neural networks (NNs) can also be utilized to predict SAR during MRI of orthopedic fixation plates based on their geometric features. The inventors have trained a neural network (NN) to predict trajectory-specific SAR of DBS leads using the distribution of the tangential component of the incident electric field along each lead trajectory. However, to date, no systems have attempted to predict local in vivo temperature changes based on individual imaging data, and with no knowledge of the implant's structure or geometry, using ML.

As the demand for MRI exams continues to grow, with an estimated 66-75% of patients with AIMDs such as DBS systems expected to require an MRI exam within 10 years of implantation, efforts to address the problem of RF heating have also increased. These efforts include modifications to the material and design of leads, implementation of new MRI transmit technology to establish a low electric field area that is specifically tailored to the patient's implanted lead, and investigation into the use of ultra-high-field and open-bore vertical scanners. Despite the ongoing efforts to mitigate RF heating in patients with AIMDs, MRI exams remain a challenge for these patients. Therefore, having real-time monitoring tools that can predict RF heating of unknown implants on a personalized basis would be highly advantageous.

Described herein is an ML-based solution to predict the temperature increase around a conductive lead. Specifically, the inventors designed a fully connected feedforward neural network to estimate the maximum change in temperature (ΔTₘₐₓ) of an implant using only the first five seconds of RF heating data obtained from the implant. The generated NN produced highly accurate predictions (R2=0.99) for the initial test dataset. Furthermore, the inventors evaluated the performance of the NN under different field strengths, MRI scanner types, and AIMDs. The results indicated that the NN could successfully predict ΔTₘₐₓ even with changes in the experimental conditions (R² up to 0.91), except for some exceptional cases where the experimentally measured ΔTₘₐₓ<0.2° C. This is important, as the configurations tested in the experiments reflected a wide range of lead trajectories and orientations with respect to the MRI electric fields, which can lead to substantial variability in the magnitude of RF heating, as observed in actual patients.

More specifically, described herein is an ML-based algorithm that predicts temperature rise in the tissue surrounding the tips of various AIMD leads after ˜3 minutes of RF exposure during MRI scans at 1.2 Tesla (T), 1.5 T, and 3 T. The prediction is based on initial data obtained in the first 5 seconds(s) of each scan. In alternative implementations, a different amount of time may be used as the basis for the prediction, such as 3 s, 4 s, 6 s, etc. The model was trained using data from a full commercial DBS system implanted in a tissue-mimicking phantom undergoing MRI at 3 T. Subsequently, the inventors evaluated the generalizability of the resulting neural network architecture by testing its ability to predict the RF heating of a DBS system during MRI scans at a different field strengths (e.g., 1.5 T), as well as a different field strength and polarization (e.g., using a 1.2 T vertical scanner), and for an unseen lead (e.g., cardiac pacemaker systems undergoing 1.5 T MRI). The results indicate that it is feasible to develop a clinically applicable tool to predict RF heating of unknown leads during MRI scans.

The rise in temperature (AT) within the tissue surrounding an implant is regulated by Penne's bioheat equation:

$$ρ c ( \partial T ( t ) ) / \partial t = ∇ ( k ( T ) ∇ T ( t ) ) + w\_bc \_b ( T\_a - T ( t ) ) + Q\_m ( t ) + SAR . \qquad Equation 1$$

The term SAR on the right side of the equation refers to the localized power dispersion around the tip of the lead of the implant. This component primarily drives RF heating in AIMD patients and exhibits the highest variability, dependent on factors such as RF frequency, lead material and trajectory within the patient, and the dielectric properties of the tissue surrounding the device. The terms k, c, ρ, and Qm in Equation 1 symbolize the thermal conductivity, heat capacity, density, and metabolic heat generation of the tissue, respectively. Although these parameters present less uncertainty compared to SAR, their variability should not be overlooked. The equation component w_b c_b (T_a−T(t)) signifies the influence of perfusion (i.e., the passage of blood through channels in an organ or tissue), a factor often omitted in ΔT estimation to widen the safety margin.

Given that these patient-specific parameters are not directly measurable, the standard approach for estimating ΔT for a specific device within a certain patient population involves executing extensive simulations to determine the affects on ΔT of thermal conductivity, heat capacity, density, metabolic heat generation, SAR, perfusion, etc. These simulations utilize body models of different sizes and tissue properties, which are representative of the whole population, and are implanted with all possible device configurations. This procedure allows for the inference of ΔT statistical properties and the setting of acceptable safety thresholds based on the aforementioned parameters.

The proposed system is based on the principle that if one could measure ΔT around the lead's tip within a small volume and for a brief period when the device is subjected to a known level of total RF power (i.e., known RMS B1+), such data could sufficiently estimate (a) the locally induced SAR, and (b) the thermal properties of the surrounding medium. This data would, in turn, solve the Penn's bioheat equation to estimate ΔT for an arbitrary pulse sequence (i.e., with a scaled SAR to account for a different B1+).

The innovative crux of the proposed system posits that a Deep Learning (DL) algorithm can be used to learn to estimate SAR, k, and c in equation 1, based on limited spatial and temporal temperature data. This signifies that once the algorithm is trained to perform such mapping on data obtained from a certain device implanted in a specific tissue-simulating environment under MRI on a particular platform, it can be utilized to predict ΔT induced by a different device, implanted in another phantom, undergoing MRI on a different platform. To test the theoretical feasibility of the proposed mapping, the inventors consolidated data from RF heating experiments conducted in lab over the past 5 years, encompassing fiber optic temperature recordings from diverse implants situated in various tissue-mimicking environments, each subjected to MRI on different platforms.

A straightforward two layer neural network (NN) was first trained to forecast RF heating after 5 minutes of imaging using 5 seconds of temperature recording from deep brain stimulation devices (Abbott 40 cm 6172 lead model, connected to 50 cm 6371 extension model, and an Infinity IPG), placed in anthropomorphic phantoms following 346 unique device configurations, undergoing MRI at a horizontal 3T scanner (Prsima, Siemens Heathineers). The NN predicted ΔT (range 0.05-8.27° C.) with an MSE of 0.3° C.² and R² of 0.99 (70% training/validation, 30% test). Subsequently, the inventors applied the same NN to forecast RF heating of three novel datasets, namely, DBS devices (N=30) at horizontal 1.5 T scanner (Siemens, Aera); DBS devices (N=28) at a vertical 1.2 T scanner (Fujifilm, OASIS), and cardiac pacemakers (N=75) with varying lead lengths (Medtronic CapSure® EPI lead 4965 15 cm and 25 cm) at 1.5 T (Siemens, Aera). As discussed in more detail below, the NN efficiently predicted corresponding ATs with MSEs of 0.5° C.², 0.01° C.², and 0.8° C.² and R² values of 0.9, 0.8, and 0.9, 45 substantially outperforming a regression model with mean MSE of 1.7° C.² and mean R² of 0.69.

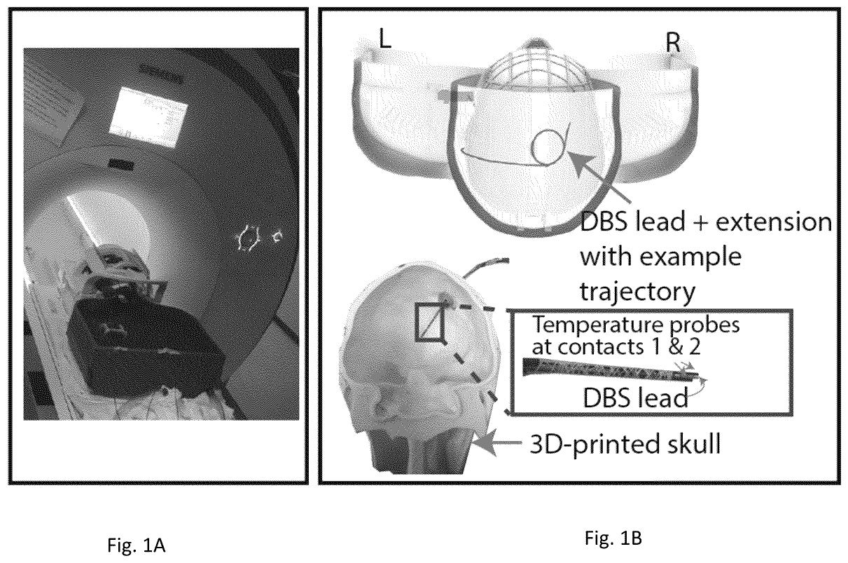

As noted, the dataset used in the analysis included 346 temperature measurements obtained from RF heating experiments performed on a full DBS system from Abbott, including a 40 centimeter (cm) lead (model 6172), a 50 cm extension (model 6371), and an Infinity-5 IPG implanted in an adult-sized anthropomorphic phantom with 346 distinct trajectories. In alternative implementations, different leads and/or implants may be used. FIG. 1A depicts an experimental setup using a 3 Tesla (T) Siemens Prisma scanner in accordance with an illustrative embodiment. In alternative embodiments, a different type of MRI scanner may be used. FIG. 1B depicts three-dimensional (3D) rendering of an anthropomorphic phantom with a DBS system in accordance with an illustrative embodiment. The DBS lead with an extension is depicted, along with an example trajectory of the lead. As shown, fiber optic temperature probes are attached to the DBS lead and used to measure the temperature at the lead-tip. Any type of temperature sensor/probe known in the art may be used. This experiment was conducted with a 3D printed skull. In alternative embodiments, a different body part may be simulated for the experiment, such as a heart, etc.

Referring to FIGS. 1A and 1B, the RF exposure was generated using a high-SAR pulse sequence (B₁⁺ rms=2.7) in a 3 T Siemens Prisma scanner (operating at 127 MHz) (Siemens Healthineers, Erlangen, Germany). The maximum temperature rise (ΔTₘₐₓ) in the surrounding gel was recorded using fiber-optic temperature probes (Osensa, Burnaby, BC, with a resolution of 0.01° Celsius (C)) attached to the lead tips. The temperature was recorded at 0.5-second increments throughout the RF exposure, which lasted for a total of 150 seconds. The ΔTₘₐₓ values were used to train the ML algorithm and served as the intended output. To ensure consistency across all experiments, the temperature was scaled such that the initial temperature (i.e., the temperature of the phantom gel at room temperature, which may vary slightly) was set to 0° C. for all experiments. The inputs to the neural network were the measured temperature values during the first five seconds of RF exposure. This input included 11 temperature values, starting from 0° C. collected at increments of 0.5° C. The first five seconds were selected as the inputs as the goal was to predict ΔTₘₐₓ around the tip of an implanted lead as quickly as possible. In alternative embodiments, a different initial time period may be used, such as 3 seconds, 4 seconds, 6 seconds, 8 seconds, 10 seconds, etc.

The total dataset was divided into a training/validation dataset (70%) and a hold-out test dataset (30%). The NN was implemented in Python (3.9.13) using Keras (2.11.0) with Tensorflow as the backend. Alternatively, a different platform may be used for the neural network or the backend. The performance of the NN was evaluated based on the coefficient of determination R-squared (R²) and the mean squared error (MSE).

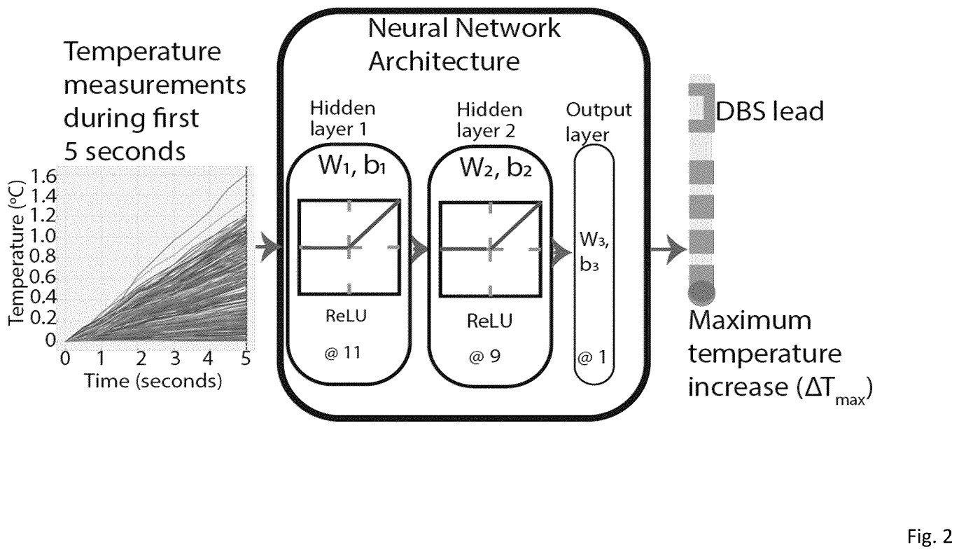

Hyperparameter optimization was performed to determine the optimal architecture of the NN. This was done using GridSearchCV with three-fold cross-validation from the scikit-learn package (1.0.2) in Python. The optimal parameters were selected based on the NN model that had the lowest MSE. The final ML algorithm included a feedforward NN with one input layer, two fully connected hidden layers with 11 and 9 hidden neurons, respectively, and an output layer. FIG. 2 depicts an architecture of a proposed neural network that includes two hidden layers with 11 and 9 hidden neurons in the first and second layer, respectively, in accordance with an illustrative embodiment. The first five seconds of experimentally measured temperature values served as the inputs to predict the maximum temperature rise. As discussed, in alternative embodiments, a different initial time period may be used, such as the first three seconds, four seconds, six seconds, ten seconds, etc. The activation function in the hidden layers was rectified linear unit (ReLU) and linear in the output layer. The output of the of the NN was the predicted ΔTₘₐₓ over the course of exposure to the RF radiation, which was then compared to the actual ΔTₘₐₓ from the RF heating experiments.

To test the generalizability of the NN, the inventors used three additional datasets from RF heating experiments with different AIMDs during MRI at different Larmor frequencies. These datasets included results from a DBS system during 1.5 T MRI (Siemens Aera closed-bore scanner, 63.6 MHZ) with 30 measurements, a DBS system during 1.2 T MRI (Fujifilm Oasis open-bore scanner, 50.4 MHZ) with 28 measurements, and cardiac pacemaker systems from Medtronic (Azure™ XT DR MRI SureScan™ IPG with CapSure® EPI lead 4965-15 cm or lead 4965-25 cm) during 1.5 T MRI with 75 measurements. The NN algorithm was applied to these new datasets to evaluate its ability to predict the RF heating of different AIMDs under different MRI conditions.

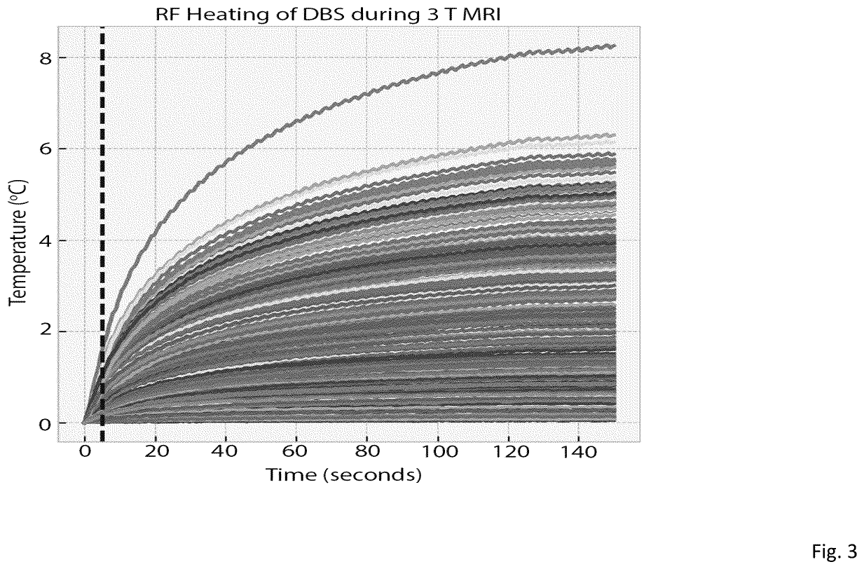

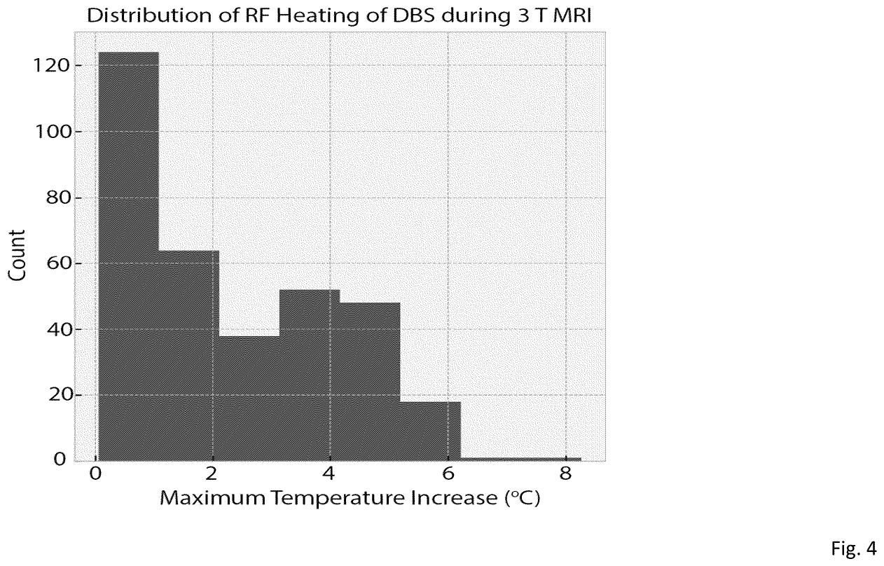

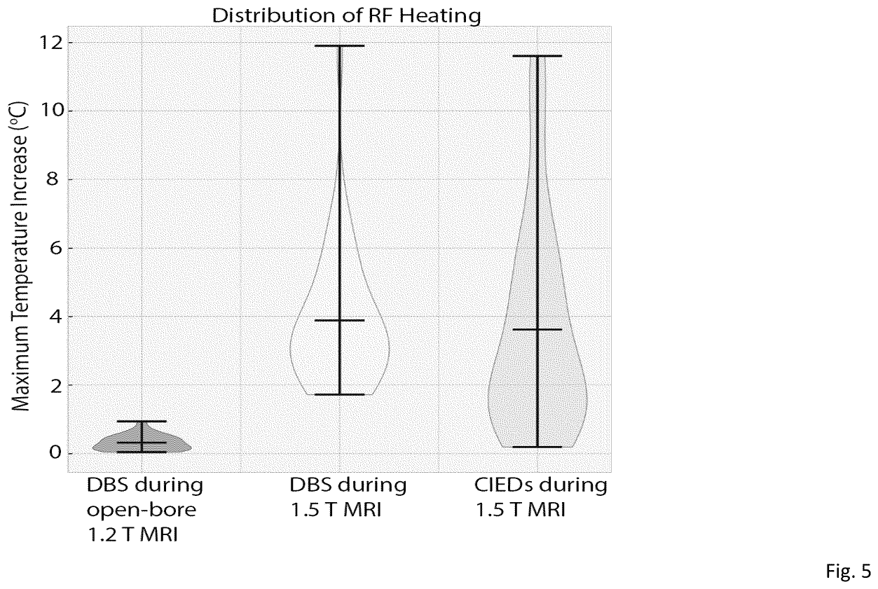

The RF heating data for 346 configurations in the initial dataset showed a mean Tₘₐₓ of 2.29±1.76° C., with a range of 0.05-8.27° C. Similarly, for the DBS system during 1.5 T MRI, the mean ΔTₘₐₓ was 3.88±2.11° C. with a range of 1.72-11.90° C. During 1.2 T MRI, the mean ΔTₘₐₓ was 0.31±0.23° C. with a range of 0.04-0.94° C. The cardiac pacemaker systems had a mean ΔTₘₐₓ of 3.62+3.10° C. with a range of 0.19-11.6° C. FIG. 3 shows the temporal profile of ΔTₘₐₓ of the 346 configurations in the initial dataset, and FIGS. 4 and 5 show the distribution of ΔTₘₐₓ for these four unique datasets.

More specifically, FIG. 3 depicts temporal profiles of temperature at the DBS lead-tip during RF exposure in accordance with an illustrative embodiment. The vertical dotted line indicates the temperature after five seconds of RF exposure. FIG. 4 depicts a distribution of ΔTₘₐₓ for 346 phantom experiments performed with a commercial DBS system in accordance with an illustrative embodiment. FIG. 5 depicts distribution of ΔTₘₐₓ for cases in the additional test datasets including RF heating of a DBS system during MRI in an open-bore scanner and MRI at 1.5 T and of CIEDs during MRI at 1.5 T in accordance with an illustrative embodiment.

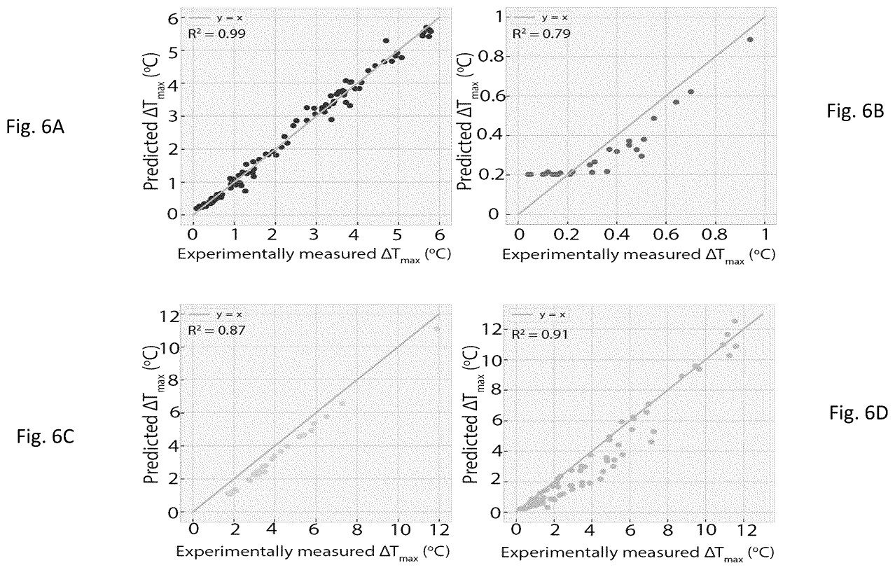

The training and validation were completed after 100 epochs. The results showed that the NN was able to accurately predict the ΔTₘₐₓ of DBS systems during 3 T MRI with a MSE of 0.3° C.² and R² of 0.99 for the hold-out test dataset. The results for the three additional test datasets were also promising, with MSEs of 0.56, 0.01, and 0.88° C.² and R² values of 0.87, 0.79, and 0.91 for RF heating of DBS systems during 1.5 T MRI and 1.2 T MRI and cardiac pacemaker systems during 1.5 T MRI, respectively. The comparison between the NN-predicted ΔTₘₐₓ and the experimentally measured ΔTₘₐₓ can be seen in FIG. 6. FIG. 6A depicts performance of the proposed neural network with the initial hold-out test dataset in accordance with an illustrative embodiment. FIG. 6B depicts performance of the proposed neural network with a DBS during MRI in an open-bore scanner at 1.2 T in accordance with an illustrative embodiment. FIG. 6C depicts performance of the proposed neural network with a DBS during MRI at 1.5 T in accordance with an illustrative embodiment. FIG. 6D depicts performance of the proposed neural network with CIEDs during MRI at 1.5 T in accordance with an illustrative embodiment.

Based on the above-discussed experiments and the resulting neural network, the inventors have developed a system that utilizes the neural network to estimate an amount of implant heating that will occur in response to imaging that is conducted using a desired pulse sequence (e.g., desired imaging power to be used during the scan and a desired duration of time of the scan). In an illustrative embodiment, an imaging pre-scan (or initial scan) is conducted on a patient with an implant. The pre-scan, which can be for a short duration such as 3 s, 5, s, 10 s, 15 s, 30 s, etc., is used to generate a series of phase images of the implant (within the patient) and the surrounding tissue. The pre-scan can also be a low power imaging scan that utilizes less energy (e.g., magnetic field strength) than a normal scan. As a result of the lower power and the short duration (e.g., up to 30 seconds) used, no implant heating occurs during the pre-scan conducted by the imaging device.

In an illustrative embodiment, the phase images generated by the imaging device (or other computing device) based on the pre-scan include both temporal information and spatial information of the implant and surrounding tissue during the pre-scan, and this information can be extracted and used by the neural network. Specifically, the system utilizes the neural network to analyze the phase images and estimate values for the variables of Equation 1 based on the analysis. These variables include SAR (e.g., as a localized power dispersion around the tip of the lead of the implant), the thermal conductivity of the tissue surrounding the implant, heat capacity of the tissue surrounding the implant, density of the tissue surrounding the implant, metabolic heat generation of the tissue surrounding the implant, and the amount of perfusion in the tissue surrounding the implant. In alternative implementations, different dielectric properties of the tissue may also be determined.

It is noted that the estimated values of the above-discussed variables are based on at least the specific location of the implant within the patient (which dictates the surrounding tissue), the type and trajectory of the implant, the frequency of the imaging device, and the power of the imaging device. As a result, the estimated values are specific to the implant, to its position within the patient, and to the machine used to perform the imaging. For different scenarios (i.e., different implant, patient, and/or imaging machine), a different pre-scan can be used to generate different phase images corresponding to the new scenario.

Upon determining values for the variables of Equation I based on the phase images that are generated during the pre-scan, the system can utilize those values to determine an amount of heating that will occur at the implant in response to a scan of normal duration and power. More specifically, the system utilizes the determined values for the variables along with desired pulse sequence values specified by the operator to determine the temperature increase in the implant that will occur during such a subsequent scan that uses the desired pulse sequence values. The pulse sequence values can include a desired power for an imaging scan (e.g., B1+=3 uT) and a desired duration for the imaging scan (e.g., 15 minutes), and these values are determined by a physician or other individual conducting the imaging scan.

As known to those of skill in the art, the desired power of the scan and the desired duration of the scan can vary based on the location of the implant, the type of implant, the patient, the goal of the imaging, etc. Similarly, the acceptable amount of temperature increase in the implant can vary based on the location of the implant in the patient. As an example, it may be acceptable to have a higher temperature increase (e.g., 20 degrees) for orthopedic implants, whereas brain implants may be limited to lower temperature increases (e.g., 5 degrees) to prevent injury. Based on these considerations, the physician can determine whether the desired imaging scan can be performed based on the estimated amount of implant heating that will occur.

In some embodiments, the proposed system can compare the estimated overall amount of change in temperature to a temperature change threshold, and if the temperature change threshold is exceeded an action can be taken. The temperature change threshold can vary depending on a type of implant and/or location of the implant within the patient. The action taken can include issuing an alert (e.g., sending a text message, sending an e-mail, activating a warning light, activating an audible alarm, etc.) such that the physician or other operator is aware of the heightened risk due to the threshold being exceeded. In another embodiment, the action taken by the system can be recommending a new imaging power (e.g., magnetic field strength) and/or imaging frequency to be used during the regular scan, where the new imaging power and/or frequency will ensure that the overall amount of change in temperature of the implant does not exceed the threshold. In another embodiment, the action taken by the system can be recommending a new imaging duration to be used during the regular (subsequent) scan, where the new imaging duration will ensure that the overall amount of change in temperature of the implant does not exceed the threshold.

In another embodiment, the system can determine an acceptable maximum power for the regular scan and/or an acceptable maximum duration for the regular scan that can be used to ensure that the regular scan does not result in implant heating that exceeds an acceptable temperature change threshold. As discussed, the temperature change threshold can vary based on the location of the implant with the patient (e.g., foot vs. head), the type of implant, etc. In another embodiment, instead of the physician initially providing both a desired duration for a scan and a desired power for the scan, the physician may provide only one of these desired values and the system can determine a maximum for the other, unprovided parameter. For example, the physician may provide a desired power for the scan, and the system can utilize the process described herein to determine a maximum duration of the scan (using the desired power) such that excessive heating of the implant does not occur. Similarly, the physician may provide a desired duration for the scan, and the system can utilize the process described herein to determine a maximum power for the scan (using the desired duration) such that excessive heating of the implant does not occur.

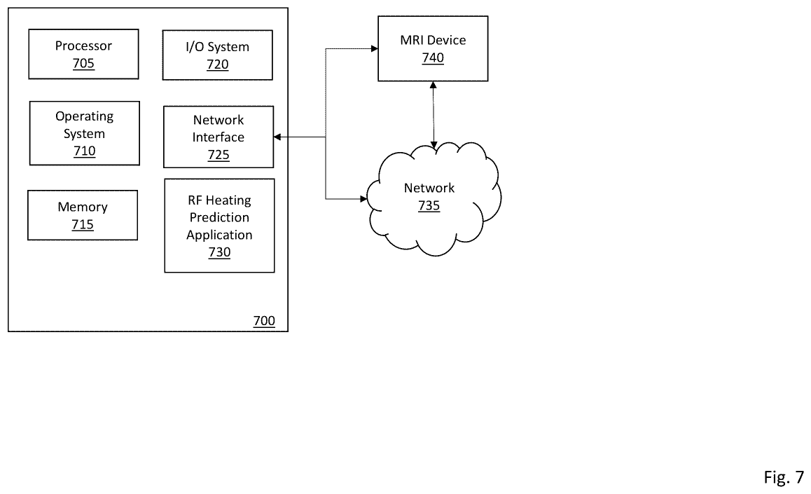

In an illustrative embodiment, any of the operations described herein can be performed by a computing system that includes a processor, a memory, a user interface, transceiver, etc. Any of the operations described herein can be stored in the memory as computer-readable instructions. Upon execution of these computer-readable instructions by the processor, the computing system performs the operations described herein. FIG. 7 is a block diagram of a computing system 700 to implement a system to predict RF heating in an implant in accordance with an illustrative embodiment.

In one embodiment, the computing system 700 is in communication with a network 735 and an MRI device 740. The computing system 700 can communicate directly with the MRI device 740 or indirectly through the network 735. In an illustrative embodiment, the MRI device 740 provides image data (e.g., one or more phase images) to the computing system 700 for use in predicting RF heating of an implant. As discussed, the phase images result from a pre-scan (or initial scan) of a patient with an implant, where the initial scan is a short duration, lower power scan that generates data for use in predicting RF heating that would result from a regular scan (i.e., having higher power and a longer duration). In an alternative embodiment, the MRI device 740 can be independent of and not in communication with the computing system 700. In such an embodiment, the computing system 700 can receive implant, image, and other data from a remote database, website, etc. Additionally, although an MRI device is depicted, in alternative embodiments a different imaging device may be used.

The computing system 700 includes a processor 705, an operating system 710, a memory 715, an input/output (I/O) system 720, a network interface 725, and an RF Heating Prediction Application 730. In alternative embodiments, the computing system 700 may include fewer, additional, and/or different components. The components of the computing system 700 communicate with one another via one or more buses or any other interconnect system. The computing system 700 can be any type of networked computing device. For example, the computing system 700 can be a smartphone, a tablet, a laptop computer, a dedicated device specific to the decoding applications, etc.

The processor 705 can be in electrical communication with and used to control any of the system components described herein. The processor 705 can be any type of computer processor known in the art, and can include a plurality of processors and/or a plurality of processing cores. The processor 705 can include a controller, a microcontroller, an audio processor, a graphics processing unit, a hardware accelerator, a digital signal processor, etc. Additionally, the processor 705 may be implemented as a complex instruction set computer processor, a reduced instruction set computer processor, an x86 instruction set computer processor, etc. The processor 705 is used to run the operating system 710, which can be any type of operating system.

The operating system 710 is stored in the memory 715, which is also used to store programs, user data, network and communications data, peripheral component data, MRI data, the RF Heating Prediction Application 730, and other operating instructions. The memory 715 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 720 is the framework which enables users and peripheral devices to interact with the computing system 700. The I/O system 720 can include one or more displays (e.g., light-emitting diode display, liquid crystal display, touch screen display, etc.), a speaker, a microphone, etc. that allow the user to interact with and control the computing system 700. The I/O system 720 also includes circuitry and a bus structure to interface with peripheral computing devices such as power sources, USB devices, data acquisition cards, peripheral component interconnect express (PCIe) devices, serial advanced technology attachment (SATA) devices, high definition multimedia interface (HDMI) devices, proprietary connection devices, etc.

The network interface 725 includes transceiver circuitry (e.g., a transmitter and a receiver) that allows the computing system to transmit and receive data to/from other devices such as the MRI device 740, other remote computing systems, servers, websites, etc. The data received from the MRI device 740 (or another source) can include a plurality of generated images, image metadata, etc. The network interface 725 enables communication through the network 735, which can be one or more communication networks. The network 735 can include a cable network, a fiber network, a cellular network, a wi-fi network, a landline telephone network, a microwave network, a satellite network, etc. The network interface 725 also includes circuitry to allow device-to-device communication such as Bluetooth® communication.

The RF Heating Prediction Application 730, which can be implemented using a neural network, can include software and algorithms in the form of computer-readable instructions which, upon execution by the processor 705, performs any of the various operations described herein such as receiving pre-scan phase images and processing the pre-scan phase images to estimate SAR as a localized power dispersion around the tip of the lead of the implant, the thermal conductivity of tissue surrounding the implant, the heat capacity of tissue surrounding the implant, the density of tissue surrounding the implant, the metabolic heat generation of the tissue surrounding the implant, the amount of perfusion through the tissue surrounding the implant, etc. Determination of values for these variables is also based on the parameters of the pre-scan that was used to generate the phase images, including the duration of the pre-scan and the power/frequency used during the pre-scan. The RF Heating Prediction Application 730 can use these variables and Equation I herein to predict an overall increase in temperature of the implant that will occur as a result of a regular scan having a desired duration and a desired power. The RF Heating Prediction Application 730 can also compare the predicted overall increase in temperature to a threshold, and generate and send an alert if the threshold will be exceeded. The RF Heating Prediction Application 730 can also determine maximum acceptable values of the power and/or duration that can be used during a regular scan to ensure that excessive implant heating does not occur. The RF Heating Prediction Application 730 can utilize the processor 705 and/or the memory 715 as discussed above. In an alternative implementation, the RF Heating Prediction Application 730 can be remote or independent from the computing system 700, but in communication therewith.

Thus, the methods and systems described herein explore the interaction between an active implantable medical device and magnetic resonance imaging (MRI) radiofrequency (RF) fields that cause excessive tissue heating of the implant. Existing methods for predicting RF heating in the presence of an implant rely on either extensive phantom experiments or electromagnetic (EM) simulations with varying degrees of approximation of the MR environment, the patient, or the implant. On the other hand, fast MR thermometry techniques can provide a reliable real-time map of temperature rise in the tissue in the vicinity of conductive implants.

As discussed, the inventors have determined that a machine learning (ML) based model is able to predict the temperature increase in the tissue near the tip of an implanted lead after several minutes of RF exposure based on only a few seconds of experimentally measured temperature values. More specifically, the proposed system employs an ultra-fast echo planar-based thermometry sequence combined with advanced deep learning algorithms to predict in-vivo RF heating of any arbitrary implant during MRI with any sequence and on any MRI platform based on a pre-scan that in one embodiment is 30 seconds or less. The inventors performed phantom experiments with a commercial deep brain stimulation (DBS) system to train a fully connected feedforward neural network (NN) to predict temperature rise after ˜3 minutes of scanning at a 3 T scanner using only the data from the first 5 seconds. The NN effectively predicted ΔTₘₐₓ−R²=0.99 for predictions in the test dataset. The model also showed potential in predicting RF heating for other various scenarios, including a DBS system at different a field strength (1.5 T MRI, R²=0.87), different field polarization (1.2 T vertical MRI, R²=0.79), and an unseen implant (cardiac lead at 1.5 T, R²=0.91). The results indicate great potential for the application of ML in combination with fast MR thermometry techniques for rapid prediction of RF heating for implants in various MR environments.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to predict heating in implants, the system comprising:
a memory configured to store one or more phase images of a medical implant that is implanted within a patient, wherein the one or more phase images include the medical implant and tissue surrounding the medical implant, and wherein the one or more phase images are based on an imaging pre-scan of the patient;
a processor operatively coupled to the memory and configured to:
analyze the one or more phase images to determine values for one or more properties of the tissue surrounding the implant; and
predict, based at least in part on the analyzed one or more phase images, a temperature increase of the medical implant that will occur during a subsequent imaging scan of the patient.

2. The system of claim 1, wherein the imaging pre-scan is conducted for 30 seconds or less.

3. The system of claim 2, wherein a first amount of power used for the pre-scan is less than a second amount of power used for the subsequent imaging scan.

4. The system of claim 1, wherein the processor predicts the temperature increase of the medical implant based at least in part on a desired power to be used during the subsequent imaging scan.

5. The system of claim 1, wherein the processor predicts the temperature increase of the medical implant based at least in part on a desired duration of the subsequent imaging scan.

6. The system of claim 1, wherein the processor analyzes the one or more phase images to determine a specific absorption rate (SAR) of the medical implant, and wherein the processor predicts the temperature increase based at least in part on the determined SAR.

7. The system of claim 1, wherein the one or more properties of the tissue include a thermal conductivity of the tissue.

8. The system of claim 1, wherein the one or more properties of the tissue include a heat capacity of the tissue.

9. The system of claim 1, wherein the one or more properties of the tissue include a density of the tissue.

10. The system of claim 1, wherein the one or more properties of the tissue include an amount of metabolic heat generation of the tissue.

11. The system of claim 1, wherein the one or more properties of the tissue include an amount of perfusion through the tissue.

12. The system of claim 1, wherein the processor utilizes a neural network to analyze the one or more phase images, and wherein the neural network includes a first hidden layer and a hidden second layer, wherein the first hidden layer includes 11 hidden neurons and the second hidden layer includes 9 hidden neurons.

13. The system of claim 1, further comprising an imaging device in communication with the processor, wherein the imaging pre-scan is performed using the imaging device, and wherein the one or more phase images are generated by the imaging device.

14. The system of claim 1, wherein the processor compares the predicted temperature increase of the medical implant to a temperature increase threshold, and wherein the processor issues an alert in response to a determination that the predicted temperature increase exceeds the temperature increase threshold.

15. The system of claim 1, wherein the processor is configured to determine, based on the analysis of the one or more phase images and a desired duration of the subsequent imaging scan, a maximum power that can be used during the subsequent imaging scan such that the temperature increase does not exceed a temperature increase threshold.

16. A method of predicting heating in implants, the method comprising:
performing, by an imaging device, an imaging pre-scan of a patient with a medical implant;
storing, in a memory of a computing device in communication with the imaging device, one or more phase images of the medical implant that is implanted within the patient, wherein the one or more phase images include tissue surrounding the medical implant, and wherein the one or more phase images result from the pre-scan of the patient;
analyzing, by a processor of the computing device, the one or more phase images to determine values for one or more properties of the tissue surrounding the implant; and
predicting, based at least in part on the analyzed one or more phase images, a temperature increase of the medical implant that will occur during a subsequent imaging scan of the patient.

17. The method of claim 16, wherein the pre-scan is conducted for 30 seconds or less, and wherein the pre-scan is conducted using a first amount of power, wherein the first amount of power is lower than a second amount of power used to conduct the subsequent imaging scan.

18. The method of claim 16, further comprising receiving at least one of a desired power and a desired duration for the subsequent imaging scan, wherein the predicted temperature increase is based on one or more of the desired power and the desired duration.

19. The method of claim 16, wherein the one or more properties of the tissue include a thermal conductivity of the tissue.

20. The method of claim 16, further comprising:
comparing, by the processor, the predicted temperature increase of the medical implant to a temperature increase threshold; and
issuing, by the processor, an alert in response to a determination that the predicted temperature increase exceeds the temperature increase threshold.
